(19) United States
(12) Patent Application Publication
(10) Pub. No.: US 2024/0082558 A1
(43) Pub. Date: Mar. 14, 2024

(54) ARTERIAL LINE PATENCY INDICATOR DEVICES, SYSTEMS, AND METHODS

(71) Applicant: Becton, Dickinson and Company, Franklin Lakes, NJ (US)

(72) Inventors: Yiping Ma, Layton, UT (US); Jonathan Karl Burkholz, Salt Lake City, UT (US)

(21) Appl. No.: 18/238,971

(22) Filed: Aug. 28, 2023

Related U.S. Application Data
(60) Provisional application No. 63/405,750, filed on Sep. 12, 2022.

(51) Int. Cl. A61M 39/10; A61M 39/20

(52) U.S. Cl. CPC A61M 39/10; A61M 39/20; A61M 2025/0003; A61M 2039/1077; A61M 2039/205

(57) ABSTRACT

A patency indicator for use with an arterial line may include a body, which may include a first end and a second end opposite the first end. The first end may include a luer adapter. The second end may be closed. The patency indicator may include a transparent portion between the first end and the second end. The patency indicator may include an air vent disposed within the body between the first end and the second end. The air vent may be configured to pass air and not blood. In response to arterial blood entering the lumen at the first end, an air pocket is configured to form between the second end and the air vent, and the transparent portion is configured to align with a leading edge of the arterial blood for visualization of the leading edge of the arterial blood.

### RELATED APPLICATIONS

This application claims the benefit of U.S. Provisional Patent Application No. 63/405,750, which was filed on Sep. 12, 2022, which is incorporated herein in its entirety.

### BACKGROUND

Arterial catheterization is a vital procedure that is used ubiquitously in the hospital setting, both in critically injured and perioperative patients. It is estimated that more than eight million arterial catheters are placed yearly in the United States. Arterial catheters can continuously and accurately measure blood pressure as well as heart rate and pulse contour to allow for immediate recognition of aberrant hemodynamic events and initiation of appropriate treatment. Arterial catheters also provide samples for blood gas analysis without the morbidity associated with repeat arterial puncture. However, one common problem occurs when a pressure sensor coupled to an arterial line stops functioning, and it is difficult to know if the pressure sensor itself has malfunctioned or if there is an occlusion in the arterial line.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a patency indicator for use with an arterial line, as well related devices, systems, and methods. In some embodiments, the arterial line may include a catheter configured for blood sampling and/or blood gas sampling. In some embodiments, the arterial line may include a pressure transducer and may be configured for blood pressure monitoring.

In some embodiments, the patency indicator for use with the arterial line may include a body. In some embodiments, the body may include a first end, which may include a luer adapter. In some embodiments, the body may include a second end opposite the first end. In some embodiments, the second end may be closed. In some embodiments, the body may include a transparent portion between the first end and the second end.

In some embodiments, the patency indicator may include a lumen extending through the first end and towards the second end. In some embodiments, the patency indicator may include an air vent disposed within the body between the first end and the second end. In some embodiments, the air vent may provide venting of the lumen and may be configured to pass air and not blood. In some embodiments, the air vent may include a hydrophobic membrane, and when the hydrophobic membrane is wetted by blood, it may lose its capacity to vent air and become a barrier to prevent atmospheric air from entering the lumen.

In some embodiments, in response to arterial blood entering the lumen at the first end, an air pocket may be configured to form between the second end and the air vent, and the transparent portion may be configured to align with a leading edge of the arterial blood for visualization of the leading edge of the arterial blood. In some embodiments, the second end may be closed such that air is prevented from exiting the second end, and the air pocket may form proximate the second end, unvented. In some embodiments, arterial blood may enter the lumen at the first end until the air vent is wetted, leaving the air pocket in the patency indicator.

In some embodiments, the leading edge of the arterial blood may pulsate in response to heart beats, which may compress air within the air pocket. In some embodiments, the pulsation of the arterial blood may indicate the arterial line distal to the patency indicator is patent. In some embodiments, the patency indicator may provide a quick, cost-effective way to assess patency of the arterial line. In some embodiments, the patency indicator may provide continuous and/or long-term monitoring of the arterial line.

In some embodiments, the luer adapter may include a luer slip adapter, which may facilitate coupling to a port of the arterial line. In some embodiments, the luer adapter may include a luer lock adapter, which may facilitate removable coupling to the port. In some embodiments, the luer lock adapter may include a collar, which may be threaded for a secure, removable fit with the port. In some embodiments, the first end may be bonded to the arterial line such that the patency indicator is permanently or non-removably coupled to the arterial line. In some embodiments, the patency indicator may be coupled to the arterial line in another suitable manner.

In some embodiments, the port of the arterial line to which the patency indicator may be coupled may include a port distal to a pressure transducer coupled to the arterial line. In some embodiments, the port of the arterial line to which the patency indicator may be coupled may include a sampling port proximal to a catheter assembly within the arterial line. In these embodiments, the patency indicator may facilitate detection of an occlusion distal to the sampling port. In other embodiments, the port of the arterial line to which the patency indicator may be coupled may be disposed on a catheter adapter of the catheter assembly. In these embodiments, the patency indicator may facilitate detection of an occlusion within the catheter assembly.

In some embodiments, the transparent portion may be constructed of a transparent material, which may facilitate visualization of the leading edge within the lumen. In some embodiments, the body, which may include an annular wall, may be entirely or partially constructed of the transparent material. In these embodiments, the transparent portion may be monolithically formed as a single unit with the first end and the second end such that an entirety of the body is transparent. In some embodiments, the transparent portion may include a lens, which may improve visualization of the leading edge within the lumen. In some embodiments, the lens may include a magnifying lens. In some embodiments, the air vent may be disposed closer to the first end than the lens is to the first end, because the arterial blood may travel slightly past the air vent prior to the air vent being wetted such that the air is trapped within the air pocket.

In some embodiments, a diameter of a first portion of the lumen between the air vent and the first end is greater than a diameter of a second portion of the lumen between the air vent and the second end. In these embodiments, due to the second portion having a narrowed diameter compared to the first portion and being elongated, the leading edge may move a larger distance in response to a heartbeat, which may facilitate visualization of the leading edge by a clinician. In some embodiments, the second portion of the lumen between the air vent and the second end may be generally cylindrical, which may facilitate visualization of movement of the leading edge in response to a heartbeat. In some embodiments, the air vent may be annular and surrounds the second portion of the lumen. In some embodiments, in response to arterial blood entering the lumen at the first end, the leading edge of the arterial blood and the air pocket may be disposed within the second portion. In some embodiments, the transparent portion may be aligned with the second portion of the lumen.

In some embodiments, an arterial line may include the catheter assembly, which may include a catheter, such as an arterial catheter configured for insertion into an artery of a patient. In some embodiments, the arterial line may include a pressure transducer. In some embodiments, the arterial line may include the patency indicator disposed between the catheter assembly and the pressure transducer. In some embodiments, the catheter assembly may include a catheter adapter, and the catheter may extend from a distal end of the catheter adapter.

In some embodiments, the port may include a luer adapter, such as, for example, a female luer adapter configured to receive the patency indicator. In some embodiments, the catheter adapter may include the port, and the patency indicator may be coupled to the port. In some embodiments, the arterial line may include a sampling port disposed between the catheter assembly and the pressure transducer, and the patency indicator may be coupled to the sampling port. In some embodiments, the sampling port may be separated from the catheter adapter by one or more extension tubes and may be configured for blood sampling.

In some embodiments, a method of detecting an occlusion may include coupling the patency indicator to the arterial line. In some embodiments, the method may include visualizing a leading edge of the arterial blood through the transparent portion in response to arterial blood entering the lumen at the first end. In some embodiments, if the clinician is able to visualize the leading edge of the arterial blood, which may pulsate in response to heart beats, through the transparent portion, the clinician may determine that a problem is likely with the pressure transducer and not with the arterial line. On the other hand, if the clinician is not able to visualize the leading edge of the arterial blood through the transparent portion and pulsation of the leading edge in response to heart beats, then the clinician may determine that there is failure to place the catheter within the artery and/or an occlusion of the arterial line.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is a schematic diagram of an example arterial line, according to some embodiments;

FIG. 1B is an upper perspective view of another example arterial line, according to some embodiments;

FIG. 2A is an upper perspective view of an example patency indicator, according to some embodiments;

FIG. 2B is a top view of the patency indicator of FIG. 2A, according to some embodiments;

FIG. 2C is a longitudinal cross-sectional view of the patency indicator of FIG. 2A coupled to an example port, illustrating an example leading edge, according to some embodiments;

FIG. 2D is a longitudinal cross-sectional view of another patency indicator, according to some embodiments;

FIG. 3A is an upper perspective view of an example patency indicator, according to some embodiments;

FIG. 3B is a view from a second end of the patency indicator of FIG. 3A, according to some embodiments;

FIG. 3C is another upper perspective view of the patency indicator of FIG. 3A, according to some embodiments;

FIG. 3D is a longitudinal cross-sectional view of the patency indicator of FIG. 3A coupled to the port, according to some embodiments.

### DESCRIPTION OF EMBODIMENTS

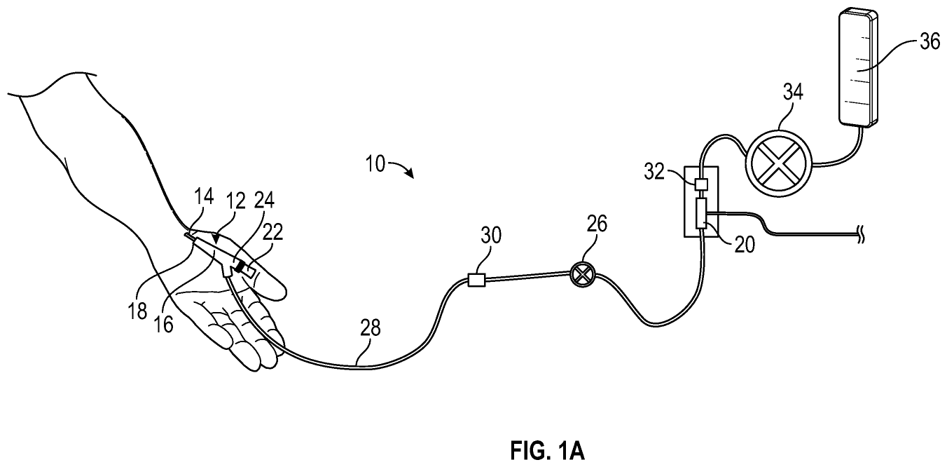

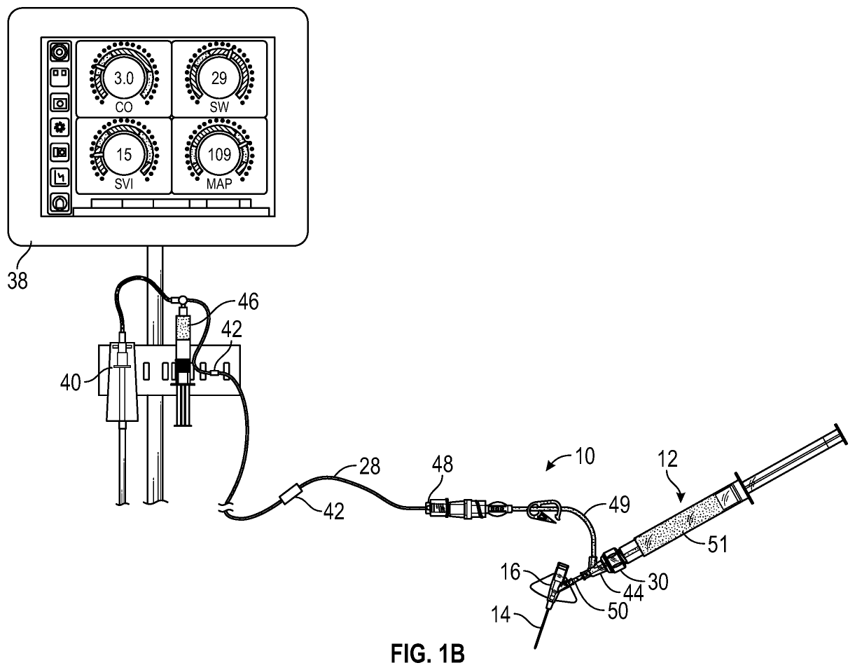

Referring now to FIG. 1A, an arterial line 10 is illustrated, according to some embodiments. In some embodiments, the arterial line 10 may include a catheter assembly 12, which may include a catheter 14, such as an arterial catheter configured for insertion into an artery of a patient. In some embodiments, the catheter assembly 12 may include a catheter adapter 16, and the catheter 14 may extend from a distal end 18 of the catheter adapter 16. In some embodiments, the arterial line 10 may include a pressure transducer 20. In some embodiments, the arterial line 10 may include a patency indicator 22 disposed between the catheter assembly 12 and the pressure transducer 20. In some embodiments, the patency indicator 22 may be in fluid communication with a fluid pathway extending through the arterial line 10.

In some embodiments, the patency indicator 22 may be coupled to a port. In some embodiments, the port may include a luer adapter, such as, for example, a female luer adapter configured to receive the patency indicator 22. In some embodiments, the port may be a port 24 of the catheter adapter 16, which may include a side port or any suitable port of the catheter adapter 16. In some embodiments, the port may be a sampling port 26 disposed between the catheter assembly 12 and the pressure transducer 20, and the patency indicator 22 may be coupled to the sampling port 26. In some embodiments, the sampling port 26 may be separated from the catheter adapter 16 by an extension tube 28, or multiple extension tubes, and may be configured for blood sampling if the patency indicator 22 is removed. In some embodiments, the extension tube 28 may be coupled to or extend from the catheter adapter 16 and/or may include a clamp 30.

In some embodiments, the pressure transducer 20 may include any suitable pressure transducer configured to pressure blood pressure known in the art. In some embodiments, the arterial line 10 may include a squeezable fast flush valve 32 and/or a slow flush valve 34. In some embodiments, the arterial line 10 may include a pressured bag of saline 36.

Referring now to FIG. 1B, a configuration of the arterial line 10 may vary, according to some embodiments. FIG. 1B is another example of the arterial line 10. In some embodiments, the arterial line 10 may be coupled to a hemodynamic monitoring system 38, which may be operatively coupled to one or more hemodynamic monitoring system sensors 40. In some embodiments, a second sampling port 42 may provide an alternate or additional port than an access connector 44 for coupling of the patency indicator 22. In some embodiments, the second sampling port 42 may be used in perioperative and surgical settings. The second sampling port 42 may be eliminated due to presence of the access connector 44, negating a need for the second sampling port 42 in some embodiments.

In some embodiments, a temporary discard sample syringe 46 may be secured to the hemodynamic monitoring system 38 along with the hemodynamic monitoring system sensors 40, if desired. In some embodiments, the extension tube 28 may be fluidically connected to the catheter assembly 12 via a connector 48, which may be coupled to another extension tube 49. In some embodiments, another extension tube 50 may be disposed between the access connector 44 and the catheter adapter 16, as may be further described in U.S. Patent App. No. 63/405,700, filed Sep. 12, 2022, entitled “INTEGRATED CATHETER SYSTEM CONFIGURED FOR BLOOD SAMPLING.” In some embodiments, a blood sampling device 51 may be coupled to the access connector 44.

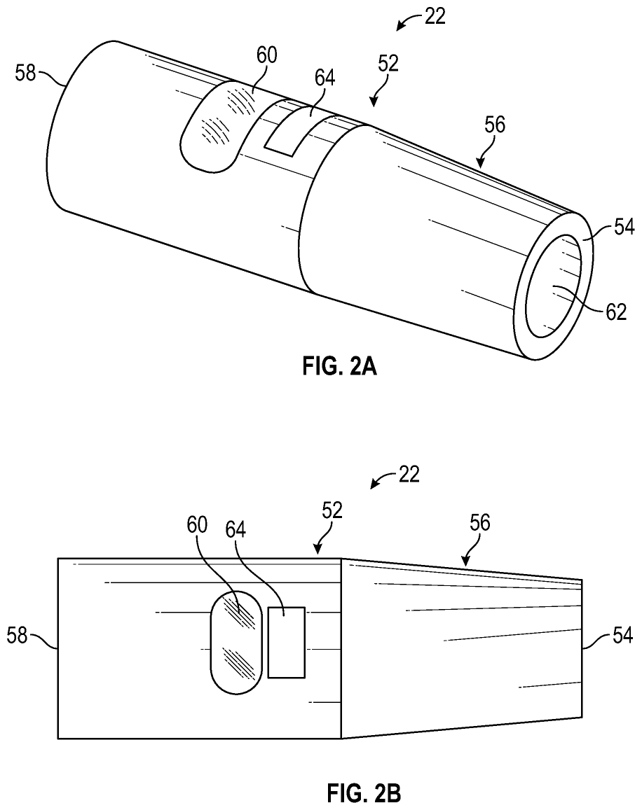

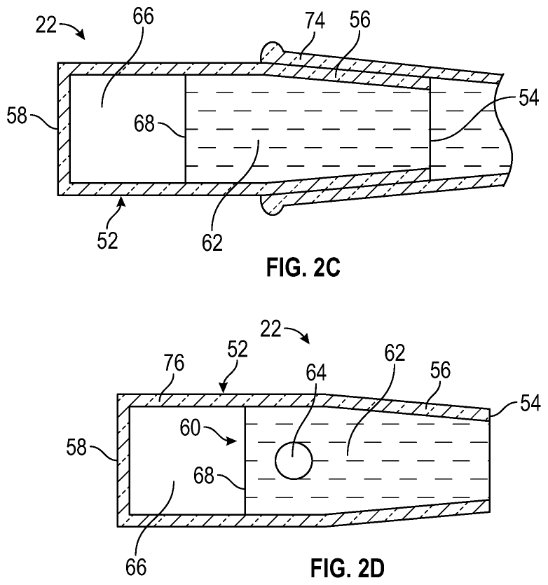

Referring now to FIGS. 2A-2C, in some embodiments, the patency indicator 22 for use with an arterial line 10 may include a body 52. In some embodiments, the body 52 may include a first end 54, which may include a luer adapter 56. In some embodiments, the body 52 may include a second end 58 opposite the first end 54. In some embodiments, the second end 58 may be closed. In some embodiments, the body 52 may include a transparent portion 60 between the first end 54 and the second end 58.

In some embodiments, the patency indicator 22 may include a lumen 62 extending through the first end 54 and towards the second end 58. In some embodiments, the patency indicator 22 may include an air vent 64 disposed within the body 52 between the first end 54 and the second end 58. In some embodiments, the air vent 64 may provide venting of the lumen 62 and may be configured to pass air and not blood. In some embodiments, the air vent 64 may include a hydrophobic membrane, and when the hydrophobic membrane is wetted by blood, it may lose its capacity to vent air and become a barrier to prevent atmospheric air from entering the lumen. In some embodiments, the air vent 64 may include any other suitable type of air vent known in the art.

In some embodiments, in response to arterial blood entering the lumen 62 at the first end 54, an air pocket 66 is configured to form between the second end 58 and the air vent 64, and the transparent portion 60 is configured to align with a leading edge 68 of the arterial blood for visualization of the leading edge 68 of the arterial blood. In some embodiments, the second end 58 may be closed such that air is prevented from exiting the second end 58, and the air pocket 66 may form proximate the second end 58, unvented. In some embodiments, arterial blood may enter the lumen 62 at the first end 54 until the air vent 64 is wetted, leaving the air pocket 66 in the patency indicator 22.

In some embodiments, the leading edge 68 of the arterial blood may pulsate in response to heart beats, which may compress air within the air pocket 66. In some embodiments, the pulsation of the arterial blood may indicate the arterial line distal to the patency indicator 22 is patent. In some embodiments, the patency indicator 22 may provide a quick, cost-effective way to assess patency of the arterial line. In some embodiments, the patency indicator 22 may provide continuous and/or long-term monitoring of the arterial line.

In some embodiments, the luer adapter may include a luer slip adapter, illustrated, for example, in FIGS. 2A-2D, which may facilitate coupling to a port of the arterial line. In some embodiments, the luer adapter may be tapered inwardly and proximate a generally cylindrical portion, which may include the second end 58. In some embodiments, the first end 54 may instead be bonded to the arterial line such that the patency indicator is permanently or non-removably coupled to the arterial line.

In some embodiments, the transparent portion 60 may include a lens, illustrated, for example, in FIGS. 2A-2B, which may improve visualization of the leading edge 68 within the lumen 62. In some embodiments, the lens may include a magnifying lens. In some embodiments, the air vent 64 may be disposed closer to the first end 54 than the lens is to the first end 54, because the arterial blood may travel slightly past the air vent 64 prior to the air vent 64 being wetted such that the air is trapped within the air pocket 66.

In some embodiments, a port 74 of the arterial line to which the patency indicator 22 may be coupled may be distal to a pressure transducer, such as, for example, the pressure transducer 20 of FIG. 1A or 1B, coupled to the arterial line. In some embodiments, the port 74 of the arterial line to which the patency indicator may be coupled may include a sampling port, such as, for example, the sampling port 26 of FIG. 1A-1B or the access connector 44 of FIG. 1B. In some embodiments, the sampling port may be proximal to a catheter assembly within the arterial line. In these embodiments, the patency indicator 22 may facilitate detection of an occlusion distal to the port 74. In other embodiments, the port 74 of the arterial line to which the patency indicator may be coupled may be disposed on a catheter adapter of a catheter assembly, such as, for example, illustrated in FIG. 1A. In these embodiments, the patency indicator 22 may facilitate detection of an occlusion within the catheter assembly.

Referring now to FIG. 2D, in some embodiments, as an alternative to the lens, the transparent portion 60 may be constructed of a transparent material, which may facilitate visualization of the leading edge 68 within the lumen 62. In some embodiments, the body 52, which may include an annular wall 76, may be entirely or partially constructed of the transparent material. In these embodiments, the transparent portion may be monolithically formed as a single unit with the first end 54 and the second end 58 such that an entirety of the body 52 is transparent.

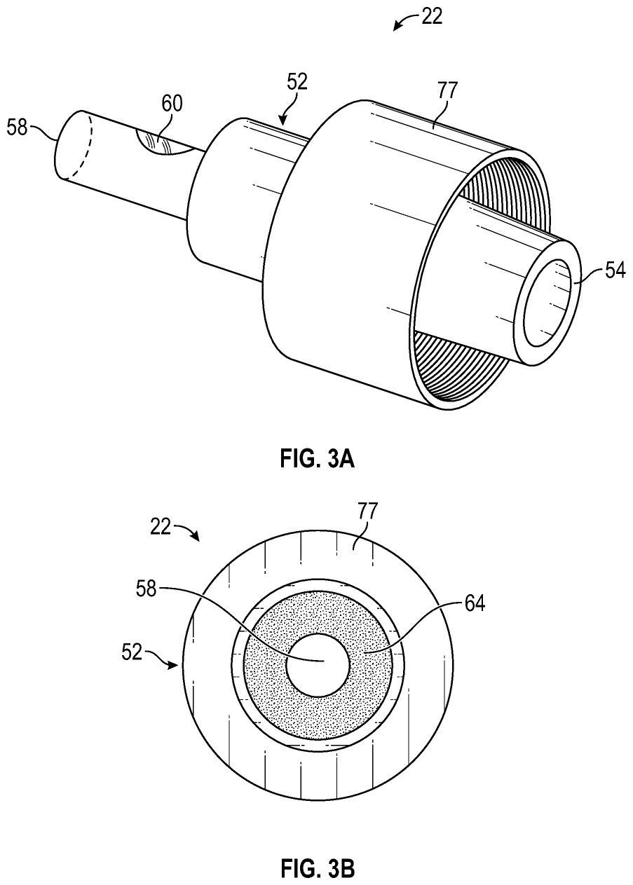

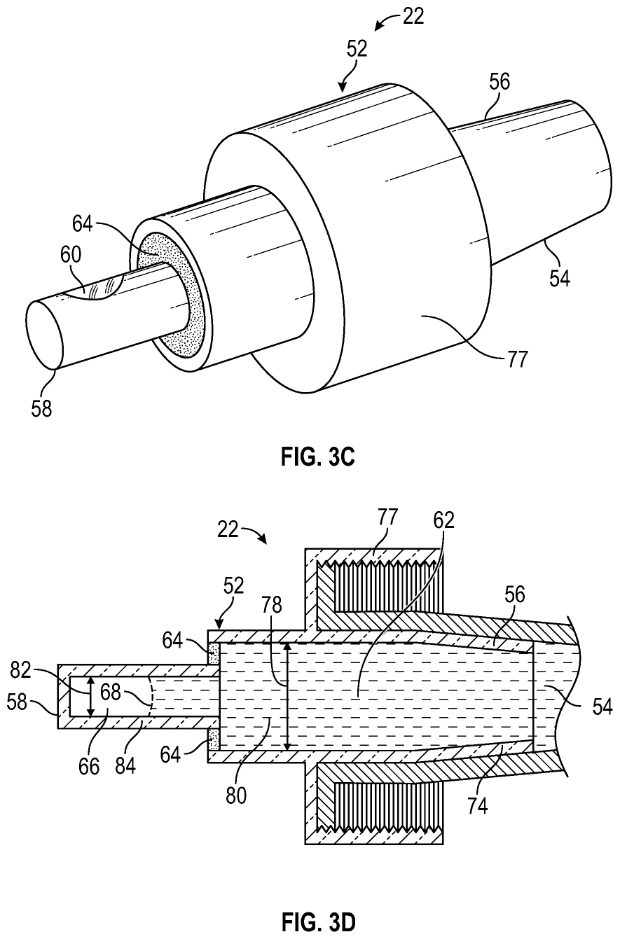

Referring now to FIGS. 3A-3B, the patency indicator 22 is illustrated, according to some embodiments. In some embodiments, the patency indicator 22 of FIGS. 3A-3D may be similar or identical to the patency indicator 22 of FIGS. 2A-2D in terms of one or more features and/or operation. In some embodiments, the luer adapter 56 may include a luer lock adapter, which may facilitate removable coupling to the port of the arterial line. In some embodiments, the luer lock adapter may include a collar 77, which may be threaded for a secure, removable fit with the port 74.

In some embodiments, a diameter 78 of a first portion 80 of the lumen 62 between the air vent 64 and the first end 54 may be greater than a diameter 82 of a second portion 84 of the lumen 62 between the air vent 64 and the second end 58. In these embodiments, due to the second portion 84 having a narrowed diameter compared to the first portion 80 and being elongated, the leading edge 68 may move a larger distance in response to a heartbeat, which may facilitate visualization of the leading edge 68 by a clinician. In some embodiments, the second portion 84 of the lumen 72 between the air vent 64 and the second end 58 may be generally cylindrical, which may facilitate visualization of movement of the leading edge 68 in response to a heartbeat. In some embodiments, the air vent 64 may be annular and surrounds the second portion 84 of the lumen 72. In some embodiments, in response to arterial blood entering the lumen 62 at the first end 54, the leading edge 68 of the arterial blood and the air pocket 66 may be disposed within the second portion 84. In some embodiments, the transparent portion 60 may be aligned with the second portion 84 of the lumen 62. In some embodiments, a portion of the body 52 forming the second portion 84 and/or a portion of the body 52 forming the first portion 80 may be generally cylindrical. In some embodiments, a diameter of the portion of the body 52 forming the second portion 84 may be less than a diameter of the portion of the body 52 forming the first portion 80.

Referring back to FIGS. 1A-1B, in some embodiments, a method of detecting an occlusion may include coupling the patency indicator 22 to the arterial line 10. In some embodiments, the method may include visualizing a leading edge of the arterial blood through the transparent portion in response to arterial blood entering the lumen of the patency indicator 22 at the first end. In some embodiments, if the clinician is able to visualize the leading edge of the arterial blood, which may pulsate in response to heart beats, through the transparent portion, the clinician may determine that a problem is likely with the pressure transducer and not with the arterial line. On the other hand, if the clinician is not able to visualize the leading edge of the arterial blood through the transparent portion and pulsation of the leading edge in response to heart beats, then the clinician may determine that there is failure to place the catheter 14 within the artery and/or an occlusion of the arterial line 10.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A patency indicator for use with an arterial line, the patency indicator comprising:
a body, comprising:
a first end, comprising a luer adapter;
a second end opposite the first end, wherein the second end is closed; and
a transparent portion between the first end and the second end;
a lumen extending through the first end and towards the second end; and
an air vent disposed within the body between the first end and the second end, wherein the air vent is configured to pass air and not blood.

2. The patency indicator of claim 1, wherein in response to arterial blood entering the lumen at the first end, an air pocket is configured to form between the second end and the air vent, and the transparent portion is configured to align with a leading edge of the arterial blood for visualization of the leading edge of the arterial blood.

3. The patency indicator of claim 1, wherein the luer adapter comprises a luer slip adapter.

4. The patency indicator of claim 1, wherein the luer adapter comprises a luer lock adapter.

5. The patency indicator of claim 1, wherein the transparent portion is monolithically formed as a single unit with the first end and the second end, wherein the body is constructed of a transparent material.

6. The patency indicator of claim 1, wherein the transparent portion comprises a lens.

7. The patency indicator of claim 1, wherein the air vent is disposed closer to the first end than the lens is to the first end.

8. The patency indicator of claim 1, wherein a diameter of a first portion of the lumen between the air vent and the first end is greater than a diameter of a second portion of the lumen between the air vent and the second end.

9. The patency indicator of claim 8, wherein the second portion of the lumen between the air vent and the second end is generally cylindrical.

10. The patency indicator of claim 8, wherein the air vent is annular and surrounds the second portion of the lumen.

11. The patency indicator of claim 8, wherein in response to arterial blood entering the lumen at the first end, the leading edge of the arterial blood and the air pocket are disposed within the second portion.

12. The patency indicator of claim 11, wherein the transparent portion is aligned with the second portion of the lumen.

13. An arterial line, comprising:
a catheter assembly, comprising a catheter;
a pressure transducer;
a patency indicator disposed between the catheter assembly and the pressure transducer, the patency indicator comprising:
a body, comprising:
a first end, comprising a luer adapter;
a second end opposite the first end, wherein the second end is closed; and
a transparent portion between the first end and the second end;
a lumen extending through the first end and towards the second end; and
an air vent disposed within the body between the first end and the second end, wherein the air vent is configured to pass air and not blood.

14. The arterial line of claim 13, wherein in response to arterial blood entering the lumen at the first end, an air pocket is configured to form between the second end and the air vent, and the transparent portion is configured to align with a leading edge of the arterial blood for visualization of the leading edge of the arterial blood.

15. The arterial line of claim 13, wherein the catheter assembly, comprises a catheter adapter, wherein the catheter extends from a distal end of the catheter adapter, wherein the catheter adapter comprises a port, wherein the patency indicator is coupled to the port.

16. The arterial line of claim 13, further comprising a sampling port disposed between the catheter assembly and the pressure transducer, wherein the patency indicator is coupled to the sampling port.

17. A method of detecting an occlusion, the method comprising:
coupling a patency indicator to an arterial line, wherein the patency indicator comprises:
a body, comprising:
a first end, comprising a luer adapter;
a second end opposite the first end, wherein the second end is closed; and
a transparent portion between the first end and the second end;
a lumen extending through the first end and towards the second end; and
an air vent disposed within the body between the first end and the second end, wherein the air vent is configured to pass air and not blood; and
in response to arterial blood entering the lumen at the first end, visualizing a leading edge of the arterial blood through the transparent portion.

18. The method of claim 17, wherein the luer adapter comprises a luer slip adapter.

19. The method of claim 17, wherein the luer adapter comprises a luer lock adapter.

20. The method of claim 17, wherein the transparent portion comprises a lens or is constructed of a transparent material.
